(19) United States
(12) Patent Application Publication
(10) Pub. No.: US 2013/0211228 A1
(43) Pub. Date: Aug. 15, 2013

(54) METHOD FOR GENERATING A MAGNETIC RESONANCE IMAGE WITH A MAGNETIC RESONANCE TOMOGRAPHY SYSTEM

(71) Applicant: DAGMAR HERING, Oberasbach (DE)

(72) Inventor: DAGMAR HERING, Oberasbach (DE)

(21) Appl. No.: 13/587,302

(22) Filed: Aug. 16, 2012

(30) Foreign Application Priority Data
Aug. 29, 2011 (DE) 102011081734.4

(51) Int. Cl. A61B 5/055

(52) U.S. Cl. CPC A61B 5/055
USPC 600/410

(57) ABSTRACT

A method for generating a magnetic resonance image with a magnetic resonance tomography system is provided. A patient is positioned in the magnetic resonance tomography system, wherein the region of the patient to be imaged in the magnetic resonance image is covered with a sound-dampening blanket. The back and/or abdomen of the patient are covered by the sound-dampening blanket, while the magnetic resonance image is generated.

### CROSS REFERENCE TO RELATED APPLICATIONS

This application claims priority of German Patent Application No. 10 2011 081 734.4 DE filed Aug. 29, 2011. All of the applications are incorporated by reference herein in their entirety.

### FIELD OF INVENTION

A method for generating a magnetic resonance image with a magnetic resonance tomography system is provided.

### BACKGROUND OF INVENTION

Magnetic resonance tomography (MRT) is a method of easily seeing inside a human body. A strong magnet and electromagnetic waves are used here to generate significant recordings of the head, the spinal column or other parts of the body. With the aid of these recordings, a distinction can be made between healthy and diseased tissue. With this type of examination, loud noises up to 100 dB develop during rapid switching of the gradient coils in magnetic resonance tomographs, said noises affecting the patient and being perceived as bothersome. This is counteracted by the patient receiving ear plugs and/or headphones for the examination in order to protect his/her hearing.

With specific clinical indications, such as for instance in the case of a slipped disk, the generation of a magnetic resonance image of a pregnant patient may be necessary. It is possible either to implement an examination of the patient herself or the fetus in the womb. In this process the patient receives ear protection, wherein the fetus in the womb or the unborn child is exposed to the loud sound of the MR examination without any protection. Different examinations have shown that the fetus in the womb responds sensitively to noise, resulting in increased movement. Motion artifacts result there from, which significantly impair the image quality when generating a magnetic resonance image.

More recent magnetic resonance tomography systems with a larger internal diameter (patient opening) have a flat gradient coil system, as a result of which sound can be more poorly attenuated than in the case of a small patient opening. In addition, the trend for quicker MR examinations with a higher image quality has a negative affect on the sound exposition, as a result of which motion artifacts in magnetic resonance images can arise more significantly.

The publication of GOWLAND, Penny; FULFORD, Jonathan: “Initial experiences of performing fetal fMRl. Experimental neurology.”, 2004 (Vol. 190), pages 22-27, ISBN ISSN 0014-4886″ discloses a method for generating a magnetic resonance image with a magnetic resonance tomography system. To prevent vibrations from being transferred to the patient via the patient couch, it is proposed to use acoustically insulating foam, by means of which the mechanical coupling is reduced.

The publication DE 10 2004 040 112 relates to a sound-absorbing element with Helmholtz resonators, which is suited to internal spaces. An acoustic non-woven material, which is embodied as a thin fabric, can be attached the plates.

The publication of COAKLEY, Fergus V. [et al.]: “Fetal MRI: A Developing Technique for the Developing Patient. American Journal of Roentgenology.”, 2004 (Vol. 182), pages 243-252, ISBN ISSN 1546-3141″ discloses a method for a fetal MR examination.

The position of rest of the patient and the fetus is important for high quality recordings so that magnetic resonance images can be created without motion artifacts.

### SUMMARY OF INVENTION

An object is to specify an improved method for generating a magnetic resonance image in respect of noise protection.

To achieve this object, a method of the type cited in the introduction is provided having the following steps:
positioning a patient in the magnetic resonance tomography system;
covering the region of the patient to be imaged in the magnetic resonance image with a sound-dampening blanket, by the back and/or abdomen of the patient being covered by the sound-dampening blanket, and
generating the magnetic resonance image.

A noise dampening takes place by a sound-dampening blanket. As a result, no movements of the fetus are excited, as a result of which motion artifacts may be prevented. The generation of interference-free magnetic resonance images is therefore possible. Moreover, a safe examination, in terms of noise protection, of a fetus or embryo is ensured in a magnetic resonance tomography system. The method may be implemented in human or animal patients.

Expediently, a body coil of the magnetic resonance tomography system is positioned on and adjusted to the patient. This positioning is used for adjustment to the respective body size of the patient.

The back or abdomen of the patient is covered by the sound-dampening blanket, preferably up to the chest. The back and abdomen of the patient are covered in order to ensure the best possible sound dampening for the fetus in the womb. A covering of the region of the patient to be examined from the stomach to the thigh is already adequate, but an even better sound protection is ensured if the blanket extends from the chest to the thigh. The relevant region is thus completely covered in order to adequately protect the fetus in the womb from sound.

A sound-dampening blanket may be made at least on one side from a sound-dampening material such as expanded rubber, open-porous foam, chloroprene rubber, plastic or artificial leather. It is advantageous if the sound-dampening material is provided on the outer surface of the sound-dampening blanket, in respect of the interior surface facing the patient. To this end, plastics which have a low magnetic susceptibility are particularly suitable. Polycarbonate or polystyrene are worth mentioning here for instance, wherein these materials have a good shelf life and processability and are non-magnetic, electrically non-conductive and biocompatible and as a result do not lead to skin irritations or suchlike. In view of the afore-cited materials being provided internally or externally on the sound-dampening blanket, combinations of several materials are naturally possible.

In an embodiment, the sound-dampening blanket may be produced at least on one side from a porous or fibrous material, for instance natural wool or felt. The cited porous or fibrous materials are particularly efficient for the absorption of sound, since they have a large inner surface. Above all, this large inner surface is expedient in the case of high frequencies which are perceived by the patient as a high tone.

With regard to the exterior and also the interior of the sound-dampening blanket, various combinations of the cited materials are possible. Advantageously provision is made for a dense material for preventing the sound transmission between the sound source and patient to the outer surface of the sound-dampening blanket The direct sound path is thus interrupted. Porous or fibrous material which dampens high frequencies is provided for the interior surface facing the patient. An optimal noise dampening and noise protection is enabled as a result.

The sound-dampening blanket may comprise a geometric surface structure, preferably trapezoidal or pyramidal, at least on one side. On account of the shaping, this effects a significant absorption of the noises generated by the magnetic resonance tomography system. It results here that the fetus in the abdomen of the patient is protected from loud sound. Furthermore, further geometric forms are possible, for instance a triangular structure, a pimple-type profile or prism-type profile, which improve absorption of noises.

It is also conceivable to provide a sound-dampening blanket with the cited materials or the geometric surface structures only in sections.

In a further embodiment, a sound-dampening blanket with an elastic collar at at least one end, preferably at both opposing ends, is provided. Penetration of sound waves generated by the magnetic resonance tomography system is thus prevented at the upper and lower end of the blanket Further possibilities in terms of preventing penetration of sound waves naturally also exist, for instance by using a belt, which can be fastened more tightly depending on the patient size or patient circumference, in order to ensure an optimal fit.

It is likewise possible here for a sound-dampening blanket comprising a Velcro® fastener on the collar to be used. This is advantageous since the sound-dampening blanket can be adjusted with no great effort depending on the patient circumference. The sound-dampening blanket may therefore be varied in terms of its size, so that aside from patients with singleton pregnancies, patients with multiple pregnancies can also be covered by means of the sound-dampening blanket.

Further, a magnetic resonance tomography system is provided, which implements the imaging method as described, having a sound-dampening blanket which dampens noises generated by the magnetic resonance tomography system during operation when applied to the back and/or abdomen of a patient.

In addition, a use of a sound-dampening material is provided, in particular a sound-dampening blanket, for dampening noise during the generation of a magnetic resonance image with a magnetic resonance tomography system by covering the back and/or abdomen of a patient by means of a sound-dampening blanket.

### BRIEF DESCRIPTION OF THE DRAWINGS

Further advantages and details result with the aid of the following exemplary embodiments and from the drawings. The drawings are schematic representations and show:

FIG. 1 a side view of a patient during a magnetic resonance image recording;

FIG. 2 shows a cutout view of a blanket for implementing the method; and

FIG. 3 shows a further exemplary embodiment of a blanket for implementing the method.

### DETAILED DESCRIPTION OF INVENTION

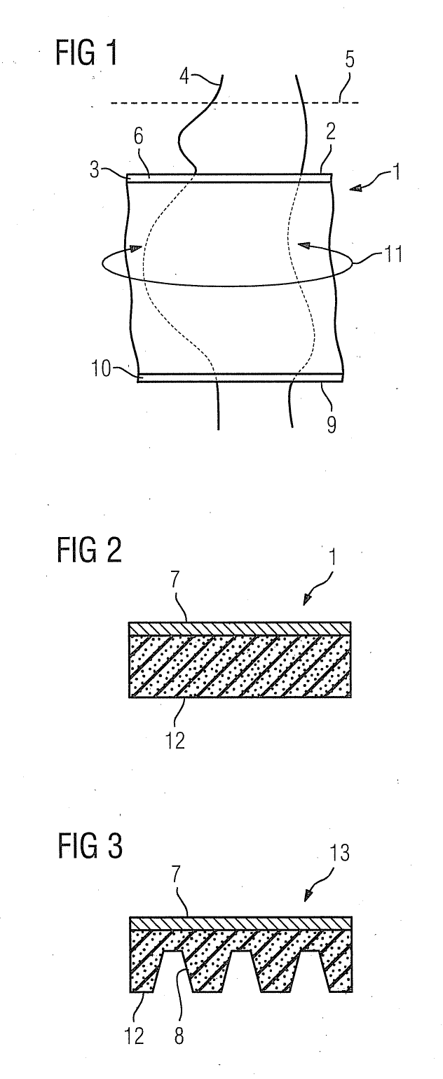

FIG. 1 shows a side view of a patient 4 in a method for generating a magnetic resonance image. A sound-dampening blanket 1 is wrapped around the examination region in the circumferential direction. The sound-dampening blanket 1 comprises an elastic collar 3, 10 at each of its opposing ends 2, 9. Sound waves generated by a magnetic resonance tomography system (not shown) are largely absorbed by the sound-dampening blanket 1. As indicated by the arrow 11, the sound-dampening blanket 1 is wrapped around the region of the patient 4 to be examined The sound-dampening blanket 1 essentially covers the abdomen of the patient 4, as indicated in FIG. 1. Alternatively, the covered region can extend from the thigh to the chest, as indicated by the dashed line 5. Consequently, a particularly good sound protection is ensured, as a result of which a fetus in the abdomen of the patient 4 is protected from loud sound.

The sound-dampening blanket 1 has a Velcro® fastener 6 on the elastic collar 3 and 10 in each instance, which is arranged over the whole length of the sound-dampening blanket 1. The Velcro® fastener 6 consists of two elements, wherein the first element has barbs and the second element straps. A detachable fastening results when connected together. This allows patients of different sizes and patients with singleton or multiple pregnancies to be covered, wherein an adjustment to different body sizes takes place by the Velcro® fastener 6, 10, as a result of which the penetration of sound waves is prevented.

The fetus in the abdomen of the patient 4 can be protected from sound waves by the sound-dampening blanket 1 with the elastic collar 3, 10 and the Velcro® fastener 6, so that no unwanted movements of the fetus are excited, which result in reduced motion artifacts. As a result, significant image recordings may be realized with a magnetic resonance tomography system, without disadvantages being expected in terms of the fetus.

FIG. 2 shows a section through the sound-dampening blanket 1, which has an exterior surface 7 and an interior surface 12. The interior surface 12 consists of natural wool, a soft material with a large inner surface, wherein a result the absorption of sound is particularly effective. This is expedient particularly with high frequencies which are perceived by the patient as a high tone. The exterior surface 7 consists of a sound-dampening material, which is chloroprene rubber in the exemplary embodiment shown. This material has a dense, but nevertheless adequately flexible surface property and is thus optimally suited to sound dampening and also to adjustment to the body of the patient 4.

FIG. 3 shows a further exemplary embodiment of a blanket in a cutout view. The sound-dampening blanket 13 consists of the same material as the blanket 1 shown in FIG. 2. Any combinations in respect of material selection for the exterior and interior surface are naturally possible. Contrary to FIG. 2, the blanket 13 has a trapezoidal surface structure 8 on the interior surface 12, which exhibits a sound-dampening effect. The geometric form of the surface structure 8 dampens the sound propagation.

While specific embodiments have been described in detail, those with ordinary skill in the art will appreciate that various modifications and alternative to those details could be developed in light of the overall teachings of the disclosure. For example, elements described in association with different embodiments may be combined. Accordingly, the particular arrangements disclosed are meant to be illustrative only and should not be construed as limiting the scope of the claims or disclosure, which are to be given the full breadth of the appended claims, and any and all equivalents thereof. It should be noted that the term “comprising” does not exclude other elements or steps and the use of articles “a” or “an” does not exclude a plurality.

1. A method of generating a magnetic resonance image with a magnetic resonance tomography system, comprising:
positioning a patient in the magnetic resonance tomography system;
covering a region of the patient to be imaged in the magnetic resonance image with a sound-dampening blanket, wherein the back and/or abdomen of the patient are covered by the sound-dampening blanket; and
generating the magnetic resonance image.

2. The method as claimed in claim 1, wherein a body coil of the magnetic resonance tomography system is positioned on and adjusted to the patient.

3. The method as claimed in claim 1, wherein the back and/or abdomen of the patient is covered by the sound-dampening blanket from thigh to chest.

4. The method as claimed in claim 1, wherein the sound-dampening blanket comprises on at least one side expanded rubber, open-porous foam, chloroprene rubber, plastic or leather.

5. The method as claimed in claim 1, wherein the sound-dampening blanket comprises on at least one side porous and/or fibrous material.

6. The method as claimed in claim 5, wherein the porous and/or fibrous material comprises natural wool or felt.

7. The method as claimed in claim 1, wherein the sound-dampening blanket comprises a geometric surface structure with trapezes or pyramids.

8. The method as claimed in claim 1, wherein the sound-dampening blanket comprises an elastic collar at at least one end.

9. The method as claimed in claim 8, wherein the sound-dampening blanket comprises two elastic collars at opposing ends.

10. The method as claimed in claim 8, wherein a Velcro® fastener is used on the sound-dampening blanket comprising a collar.

11. A magnetic resonance tomography device, comprising:
a sound-dampening blanket which dampens noises generated by the magnetic resonance system during operation, wherein the sound-dampening blanket is placeable on the back and/or abdomen of a patient.

12. The magnetic resonance tomography system as claimed in claim 11, wherein the sound-dampening blanket comprises at least on one side a sound-dampening material, which is expanded rubber, open-porous foam, chloroprene rubber, plastic or artificial leather.

13. The magnetic resonance tomography system as claimed in claim 11, wherein the sound-dampening blanket comprises at least on one side a porous and/or fibrous material.

14. The magnetic resonance tomography system as claimed in claim 13, wherein the porous and/or fibrous material comprises natural wool or felt.

15. A use of a sound-dampening blanket for dampening noise when generating a magnetic resonance image with a magnetic resonance tomography system by covering the back and/or abdomen of a patient by the sound-dampening blanket.
